SEQUENCE LISTING (6 sequences)

SEQ ID NO: 1 - DNA, 253 bp, Artificial Sequence
  16S rRNA gene sequences
tacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 2 - DNA, 253 bp, Artificial Sequence
  16S rRNA gene sequences
tacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacagg

SEQ ID NO: 3 - DNA, 1022 bp, Artificial Sequence
  16S rRNA gene sequences
agctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgactcttaaccgagtctttccttcgggacagaggagacaggtggtgcatggttgtcgtcagctcgtgtcgtgagatgttgggttaagtcccgcaacgagcgcaacccctatccccagtagccagca

SEQ ID NO: 4 - DNA, 1192 bp, Artificial Sequence
  16S rRNA gene sequences
aacctattattgaaacttcggtcgatttaatttgtttctagtggcggacgggtgagtaacgcgtgggtaacctgccctatacagggggataacaaccagaaatggttgctaataccgcataagcgcacaggaccgcatggtccagtgtgaaaaactccggtggtataggatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgatccgtaacgggatctttccttcgggacaggggagacaggtggtgcatggttgtcgtcagctcgtgtcgtgagatgttgggttaagtcccgcaacgagcgcaacccctatccccagtagccagcggtttggccgggcactctgaggagactgccagggataacctggaggaaggcggggatgacgtcaaatcatcatgccccttatgatttgggctacacacgtgctacaatggcgtaaacaaagggaagcgagcctgcgaaggtaa

SEQ ID NO: 5 - DNA, 1140 bp, Artificial Sequence
  16S rRNA gene sequences
ctgggggataacagccagaaatgactgctaataccgcataagcgcacagaaccgcatggttccgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgcagtggtgggcactctgaggagactgccagggataacctggaggaaggcggggatgacgtcaaatcatcatgccccttatgatttgggctacacacgtgctacaatggcgtaaacaaagggaagcgaacccgcgagggtgggcaaatctcaaaaataacgtcccagttc

SEQ ID NO: 6 - DNA, 1184 bp, Artificial Sequence
  16S rRNA gene sequences
actttattgaaacttcggtggatttaatttgtttctagtggcggacgggtgagtaacgcgtgggtaacctgccttatactgggggataacagccagaaatgactgctaataccgcataagcgcacagaaccgcatggttccgtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgtaatggtgggcactctgaggagactgccagggataacctggaggaaggcggggatgacgtcaaatcatcatgccccttatgatttgggctacacacgtgctacaatggcgtaaacaaagggaagcgaacccgcga

(19) United States
(12) Patent Application Publication
(10) Pub. No.: US 2024/0261342 A1
(43) Pub. Date: Aug. 8, 2024

(54) BACTERIAL STRAINS FOR USE IN PREVENTING, TREATING AND/OR REDUCING SYMPTOMS ASSOCIATED WITH A RHINOVIRUS-INDUCED COLD INFECTION

(71) Applicant: International N&H Denmark APS, KGS. LYNGBY (DK)

(72) Inventors: Ashley Ann Hibberd, New Century, KS (US); Liisa Lehtoranta, Kantvik (FI); Markus Lehtinen, Kantvik (FI)

(21) Appl. No.: 18/566,194

(22) PCT Filed: May 31, 2022
(86) PCT No.: PCT/EP2022/064745

§ 371 Date: Dec. 1, 2023

Related U.S. Application Data
(60) Provisional application No. 63/195,289, filed on Jun. 1, 2021.

(30) Foreign Application Priority Data
Sep. 30, 2021 (EP) 21200118.4

(51) Int. Cl. A61K 35/742; A61P 31/16; A61P 37/04; C12N 1/20; C12R 1/145

(52) U.S. Cl. CPC A61K 35/742; A61P 31/16; A61P 37/04; C12N 1/205; C12R 2001/145

(57) ABSTRACT

This invention relates to bacterial strains of the genus Blautia, compositions comprising bacterial said bacterial strains as well as methods and uses of said strains and compositions as a medicament, in particular for preventing, treating and/or reducing symptoms associated with a rhinovirus-induced common cold infection in a subject in need thereof.

### FIELD OF THE INVENTION

This invention relates to new bacterial strains of the Genus Blautia and compositions comprising said strains. The invention also relates to the use of said strains and compositions for preventing, treating and/or reducing symptoms associated with a rhinovirus-induced common cold infection in a subject in need thereof. The invention further relates to method of preventing, treating and/or reducing symptoms associated with a rhinovirus-induced common cold infection in a subject in need thereof, comprising treating said subject with a treatment regimen comprising administering a composition comprising an effective amount of bacteria of the genus Blautia.

### BACKGROUND OF THE INVENTION

Common cold is a heterogeneous group of mostly mild upper respiratory tract illnesses that have an impact on our daily lives. Well-known symptoms include, but are not limited to, nasal congestion, runny nose, sore throat, cough, headache, and fever that reduce quality of life and lead to absence from work, school, and day-care. From the perspective of the society, common cold has a significant economic impact on healthcare costs and lost productivity (Heikkinen and Jarvinen 2003).

Cold and flu or upper respiratory tract infections (URTI) are typically of viral origin. Different types of viruses can cause common cold like symptoms, however, rhinoviruses (RV) (30-50%), coronaviruses (10-15%), and influenza (5-15%) together account for most of the common cold episodes (Heikkinen and Jarvinen 2003; Jacobs et al. 2013). Prevalence of common cold is high, adults experience in average 3-5 episodes yearly (Monto 2002). Children tend to have more frequently infections than adults and the elderly due to yet maturing immune system (Monto, 2002). The duration of the infection and presentation of symptoms, i.e. illness, varies between individuals (Chen et al., 2015).

Cold and flu is usually a self-limiting illness (will pass by without medication) confined to the respiratory tract. Viral infection modulates the function of the immune system and in some cases common cold episode precedes bacterial infections in the middle-ear, chest tube, and nasal cavity. Therefore, decreasing the risk and length of common cold may also decrease the risk of developing more severe diseases (Winther 2011).

Prophylactic vaccines are available in the developed countries against yearly influenza virus episodes, but this is not the case for other cold viruses like RVs. Current treatments, including nasal decongestants and anti-inflammatory drugs are focused against alleviation of the symptoms, but do not have benefit of decreasing the chance of catching a cold. Limitations for available therapies in the treatment of cold and flu have elevated the attention to search for preventative approaches on reducing cold and flu incidence and severity of symptoms.

Recent studies associating microbiota and immune system function have provided opportunities in microbiota modulation and bacterial therapeutics in managing infections. Further, the studies have associated gut and airway microbiota with upper and lower respiratory tract health and immunity (Lee et al., 2019; Wypych et al., 2019), but no study so far has shown a link between specific bacteria and modulation of rhinovirus or common cold infection.

Meta-analyses of human studies indicate that the consumption of probiotics, typically lactobacilli or bifidobacteria, may offer a safe and convenient way to reduce incidence of cold and flu and severity of symptoms (Vouloumanou et al. 2009; Hao et al. 2015; Kang et al. 2013). There is, however, still a need to find other ways, including other bacteria, capable of reduce incidence of cold and flu and severity of symptoms.

### OBJECT OF THE INVENTION

In order to overcome the current challenges presented by the rhinovirus-induced common cold infections, the inventors have isolated and have shown that the new bacteria of the genus Blautia can be used in preventing, treating and/or reducing symptoms associated with the rhinovirus-induced common cold infection in subjects in need thereof.

It is therefore an object of the present invention to provide bacteria of the genus Blautia, a method, as well as compositions comprising such bacterial strains, to be used in preventing, treating and/or reducing symptoms associated with a rhinovirus-induced common cold infection in a subject in need thereof.

### SUMMARY OF THE INVENTION

In one aspect, the invention relates to a bacterial strain of the Genus Blautia, wherein said strain has a 16S ribosomal RNA sequence with at least 98.8%, at least 99% or at least 99.5% sequence identity to 16S ribosomal RNA SEQ ID NO: 3.

In another aspect, the present invention relates to a bacterial strain of the Genus Blautia, wherein said strain has a 16S ribosomal RNA sequence with at least 97.6%, at least 98% or at least 99% sequence identity to 16S ribosomal RNA SEQ ID NO: 4.

In another aspect, the present invention relates to a bacterial strain of the Genus Blautia, wherein said strain has a 16S ribosomal RNA sequence with at least 99.95% sequence identity to 16S ribosomal RNA SEQ ID NO: 5.

In another aspect, the present invention relates to a bacterial strain of the Genus Blautia, wherein said strain has a 16S ribosomal RNA sequence with at least 99.9% sequence identity to 16S ribosomal RNA SEQ ID NO: 6.

In a further aspect, the present invention relates to a bacterial strain of the genus Blautia, wherein the strain was deposited with the DSMZ on 9 Mar. 2021 under accession number DSM 33834 or has at least 80% nucleotide sequence identity to the nucleotide sequence of said deposited strain.

In a further aspect, the present invention relates to a bacterial strain of the genus Blautia, wherein the strain was deposited with the DSMZ on 10 Feb. 2021 under accession number DSM 33790 or has at least 80% nucleotide sequence identity to the nucleotide sequence of said deposited strain.

In a further aspect, the present invention relates to a bacterial strain of the genus Blautia, wherein the strain was deposited with the DSMZ on 10 Feb. 2021 under accession number DSM 33791 or has at least 80% nucleotide sequence identity to the nucleotide sequence of said deposited strain.

In yet a further aspect, the present invention relates to a bacterial strain of the genus Blautia, wherein the strain was deposited with the DSMZ on 9 Mar. 2021 under accession number DSM 33833 or has at least 80% nucleotide sequence identity to the nucleotide sequence of said deposited strain.

In yet a further aspect, the present invention relates to a use of a bacterial strain of the genus Blautia for inducing the production of interleukins IL-12 and/or IL-10.

In a further aspect, the present invention relates to a bacterial strain of the genus Blautia for use as a medicament

In yet a further aspect, the present invention relates to a bacterial strain of the genus Blautia for use in preventing, treating and/or reducing symptoms associated with a rhinovirus-induced common cold infection in a subject in need thereof.

In yet a further aspect, the present invention relates to a composition comprising bacteria of the genus Blautia for use in preventing, treating and/or reducing symptoms associated with a rhinovirus-induced common cold infection in a subject in need thereof.

In another aspect, the present invention relates to a method of preventing, treating and/or reducing symptoms associated with a rhinovirus-induced common cold infection in a subject in need thereof, comprising treating said subject with a treatment regimen comprising administering a composition comprising an effective amount of bacteria of the genus Blautia.

### Advantages

It was surprisingly found by the present inventors that the use of bacterial strains of the genera Blautia and compositions comprising said bacterial strains can be used for preventing, treating and/or reducing symptoms associated with a rhinovirus-induced common cold infection in a subject in need thereof.

### DESCRIPTION OF FIGURES

FIG. 1. Abundance of Blautia in fecal samples at clinical study day 0 from five cohorts.

FIG. 2. Scatterplot with regression line depicting the abundance of Blautia in fecal samples at study 0 against the total daily symptom score from clinical study days 1-5.

FIG. 3. Spearman correlation analysis depicting the abundance of Blautia sp. in fecal samples at study 0 against the total daily symptom score from clinical study days 1-5. Spearman rho (p) and p-values are shown for study days with significant (p<0.05) or trending (p<0.1) correlations.

### SEQUENCE LISTING

A Sequence Listing, comprising SEQ ID NOs: 1-6, is attached and incorporated herein by reference in its entirety.

### DETAILED DISCLOSURE OF THE INVENTION

The detailed aspects of this invention are set out below. In part some of the detailed aspects are discussed in separate sections. This is for ease of reference and is in no way limiting. All the embodiments described below are equally applicable to all aspects of the present invention unless the context specifically dictates otherwise.

### Bacteria

The bacterial strains of the present invention are selected from bacterial strains of the genera Blautia. Preferably the bacterial strains of the present invention are of the species Blautia obeum and Blautia faecis. In particular, the bacterial strains are chosen from the strains having SEQ ID No 3, SEQ ID No 4, SEQ ID No 5 or SEQ ID No 6 as described in the present invention. More particularly, the bacterial strains are chosen from the strains Blautia obeum P75_3586 (DGCC13628), Blautia obeum P22_1012 (DGCC13627), Blautia faecis P73_3472 (DGCC13629) and Blautia faecis P129_7565 (DGCC13626). The strains can be used individually or in combination and, for example in food products, food ingredients, dietary supplements or in pharmaceutical acceptable compositions or formulations.

Strain Blautia obeum P22_1012 (DGCC13627) was registered at the the DSMZ (Leibniz-Institut DSMZ—Deutsche Sammlung von Mikroorganismen und Zellkulturen GmbH, Inhoffenstr. 7B D-38124) under deposit number DSM33790, on 10 Feb. 2021.

Strain Blautia faecis P73_3472 (DGCC13629) was registered at the the DSMZ (Leibniz-Institut DSMZ—Deutsche Sammlung von Mikroorganismen und Zellkulturen GmbH, Inhoffenstr. 7B D-38124) under deposit number DSM33791, on 10 Feb. 2021.

Strain Blautia obeum P75_3586 (DGCC13628) was registered at the the DSMZ (Leibniz-Institut DSMZ—Deutsche Sammlung von Mikroorganismen und Zellkulturen GmbH, Inhoffenstr. 7B D-38124) under deposit number DSM33834, on 9 Mar. 2021.

Strain Blautia faecis P1297565 (DGCC13626) was registered at the the DSMZ (Leibniz-Institut DSMZ—Deutsche Sammlung von Mikroorganismen und Zellkulturen GmbH, Inhoffenstr. 7B D-38124) under deposit number DSM33833, on 9 Mar. 2021.

The conditions for cultivation of the strains are as follows:
Medium: YCFAC (Yeast Casitone Fatty Acids Agar with Carbohydrates).
Incubation time: minimal growth in 24 hours.
Temperature: 37° C.
Incubation conditions on agar plate—plates incubated at 37° C. in anaerobic conditions.

Preferably the bacterial strains to be used in the present invention are bacterial strains which are generally recognised as safe and, which are preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

In one embodiment, the present invention relates to a bacterial strain of the Genus Blautia, wherein said strain has a 16S ribosomal RNA sequence with at least 98.8%, at least 99% or at least 99.5% sequence identity to 16S ribosomal RNA SEQ ID NO: 3.

In a particular embodiment, the present invention relates to a bacterial strain of the Genus Blautia, wherein said strain has a 16S ribosomal RNA sequence identical to SEQ ID NO: 3.

In another embodiment, the present invention relates to a bacterial strain of the Genus Blautia, wherein said strain has a 16S ribosomal RNA sequence with at least 97.6%, at least 98% or at least 99% sequence identity to 16S ribosomal RNA SEQ ID NO: 4.

In a more specific embodiment, the bacterial strain of the Genus Blautia has a 16S ribosomal RNA sequence identical to SEQ ID NO: 4. (claim 4)

In a further embodiment, the present invention relates to a bacterial strain of the Genus Blautia, wherein said strain has a 16S ribosomal RNA sequence with at least 99.95% sequence identity to 16S ribosomal RNA SEQ ID NO: 5.

In a specific embodiment, the bacterial strain of the Genus Blautia has a 16S ribosomal RNA sequence identical to SEQ ID NO: 5.

In another embodiment, the present invention relates to a strain of the Genus Blautia, wherein said strain has a 16S ribosomal RNA sequence with at least 99.9% sequence identity to 16S ribosomal RNA SEQ ID NO: 6.

In a specific embodiment, the bacterial strain has a 16S ribosomal RNA sequence identical to SEQ ID NO: 6.

In a further embodiment, the present invention relates to a bacterial strain of the genus Blautia, wherein the strain was deposited with the DSMZ on 9 Mar. 2021 under accession number DSM 33834 or has at least 80% nucleotide sequence identity to the nucleotide sequence of said deposited strain. More specifically, said bacterial strain of the genus Blautia has a 16S ribosomal RNA sequence with at least 98.8%, at least 99% or at least 99.5% sequence identity to 16S ribosomal RNA SEQ ID NO: 3. In a more specific embodiment, the bacterial strain of the genus Blautia is strain deposited with the DSMZ on 9 Mar. 2021 under accession number DSM 33834.

In yet a further embodiment, the present inventions relates to a bacterial strain of the genus Blautia, wherein the strain was deposited with the DSMZ on 10 Feb. 2021 under accession number DSM 33790 or has at least 80% nucleotide sequence identity to the nucleotide sequence of said deposited strain. More specifically, said bacterial strain of the genus Blautia has a 16S ribosomal RNA sequence with at least 97.6%, at least 98% or at least 99% sequence identity to 16S ribosomal RNA SEQ ID NO: 4. In a more specific embodiment, the bacterial strain of the genus Blautia is strain deposited with the DSMZ on 10 Feb. 2021 under accession number DSM 33790.

In another embodiment, the present invention relates to a bacterial strain of the genus Blautia, wherein the strain was deposited with the DSMZ on 10 Feb. 2021 under accession number DSM 33791 or has at least 80% nucleotide sequence identity to the nucleotide sequence of said deposited strain. More specifically, said bacterial of the genus Blautia has a 16S ribosomal RNA sequence with at least 99.95% sequence identity to 16S ribosomal RNA SEQ ID NO: 5. In a more specific embodiment, the bacterial strain of the genus Blautia is strain deposited with the DSMZ on 10 Feb. 2021 under accession number DSM 33791.

In a further embodiment, the present invention also relates to a bacterial strain of the genus Blautia, wherein the strain was deposited with the DSMZ on 9 Mar. 2021 under accession number DSM 33833 or has at least 80% nucleotide sequence identity to the nucleotide sequence of said deposited strain. More specifically, said bacterial strain of the genus Blautia has a 16S ribosomal RNA sequence with at least 99.9% sequence identity to 16S ribosomal RNA SEQ ID NO: 6. In a more specific embodiment, the bacterial strain of the genus Blautia is strain deposited with the DSMZ on 9 Mar. 2021 under accession number DSM 33833.

To resist rhinovirus and other respiratory tract infections like influenza, parainfluenza, coronaviruses, adenoviruses, respiratory syncytial virus etc, it is important to boost anti-viral immunity. Interleukin (IL)-12 is a key cytokine, i.e., mediator of immune function, to induce activation of innate and adaptive immune functions in viral infections. More closely, IL-12 activates innate immune cells, like, macrophages to enhance their functions and is a critical cytokine secreted by dendritic cells to induce T helper type 1 (Th1) cells that are needed in response to viral infections in general and also rhinovirus infections in particular.

While immune system is activated to fight respiratory viruses, it is also important to manage and control excess inflammation. Higher inflammation is shown to correlate with higher symptom scores during viral infections, for example when inflammatory IL-8 is used as a marker. IL-10 is a cytokine that controls the inflammatory response during infection and can thus counteract effects of the inflammatory cytokines.

As shown by the present invention, the bacterial strains according to the present invention induce the production of interleukins IL-12 and/or IL-10. Therefore, the present invention also relates to a use of a bacterial strain of the genus Blautia for inducing the production of interleukins IL-12 and/or IL-10.

In another aspect of the present invention, the interleukins IL-12 and/or IL-10 induce an immunoboosting effect.

In another aspect, the interleukins IL-12 and/or IL-10 induce a T-helper 1 (Th1) type immunostimulation and/or an anti-inflammatory effect.

The present invention also relates to bacterial strains of the genus Blautia for use as a medicament.

In a particular aspect, when the bacterial strain of the genus Blautia is used as a medicament, the bacterial strain induces the production of interleukins IL-12 and/or IL-10. In a particular aspect, the interleukins IL-12 and/or IL-10 induce an immunoboosting effect. In a more particular aspect, the interleukins IL-12 and/or IL-10 induce a T-helper 1 (Th1) type immunostimulation and/or an anti-inflammatory effect.

The present invention further relates to a bacterial strain of the genus Blautia for use in preventing, treating and/or reducing symptoms associated with a rhinovirus-induced common cold infection in a subject in need thereof.

In one embodiment, the term “subject”, as used herein, means a mammal, including for example humans. In one embodiment the subject is a human. In one embodiment the subject is female. In one embodiment the subject is male.

In particular, the symptoms associated with a rhinovirus-induced common cold infection are sneezing, nasal obstruction, nasal discharge, sore throat, cough, headache, chilliness and malaise.

The strains according to the present invention are strains of the genus Blautia. In particular, the present invention relates to strain of the genus Blautia deposited with DSMZ on 10 Feb. 2021 under accession number DSM 33790. In another aspect of the present invention, the strains of the present invention belong to the species Blautia obeum, such as strain deposited with DSMZ on 9 Mar. 2021 under accession number DSM 33834 and/or Blautia faecis, such as strain deposited with DSMZ on 10 Feb. 2021 under accession number DSM 33791 and strain deposited with DSMZ on 9 Mar. 2021 under accession number DSM 33833.

In a particular embodiment according to the present invention, the bacterial strains are live bacteria.

### Compositions

While it is possible to administer strains of the genus Blautia alone according to the present invention (i.e., without any support, diluent or excipient), said strains are typically administered on or in a support as part of a product, in particular as a component or at least as one of the components of a composition, a dietary supplement, a nutritional supplement, a food product or a pharmaceutical acceptable composition or formulation. These products typically contain additional components well known to those skilled in the art.

In one embodiment, the present invention relates to a composition comprising bacteria of the genus Blautia for use as a medicament.

In one embodiment, the present invention relates to a composition comprising bacteria of the genus Blautia for use in preventing, treating and/or reducing symptoms associated with a rhinovirus-induced common cold infection in a subject in need thereof.

In a particular embodiment, the symptoms associated with a rhinovirus-induced common cold infection are sneezing, nasal obstruction, nasal discharge, sore throat, cough, headache, chilliness and malaise.

In a particular embodiment, the present invention relates to a composition comprising the strain deposited with DSMZ on Feb. 10, 2021 under accession number DSM 33790.

In a particular embodiment, the present invention relates to a composition comprising bacteria of the species Blautia obeum and/or Blautia faecis.

In a particular embodiment, the present invention relates to a composition comprising strain deposited with DSMZ on 9 Mar. 2021 under accession number DSM 33834.

In a particular embodiment, the present invention relates to a composition comprising strain deposited with DSMZ on 10 Feb. 2021 under accession number DSM 33791.

In another particular embodiment, the composition according to the present invention comprises strain deposited with DSMZ on 9 Mar. 2021 under accession number DSM 33833.

In another particular embodiment, the bacteria present in the composition according to the present invention are live bacteria.

The composition according to the present invention can be presented in different forms, such as a food product, food ingredient, a dietary supplement or a pharmaceutical acceptable composition or formulation.

In a further embodiment, the composition according to the present invention is encapsulated.

In another embodiment, the present invention relates to a method of preventing, treating and/or reducing symptoms associated with a rhinovirus-induced common cold infection in a subject in need thereof, comprising treating said subject with a treatment regimen comprising administering a composition comprising an effective amount of bacteria of the genus Blautia.

In one embodiment, the treatment regimen according to the present invention comprises oral administration of the composition. In another embodiment, the treatment regimen comprises rectal administration of the composition.

According to the present invention, the composition used in the method of preventing, treating and/or reducing symptoms associated with a rhinovirus-induced common cold infection comprises one or more of the strains of the genus Blautia as described in the present invention.

In a particular embodiment the treatment regimen according to the present invention comprises administering the composition for at least 1 week. In another embodiment, the treatment regimen comprises administering the composition for at least 2 weeks. In another embodiment, the treatment regimen comprises administering the composition at least two times per week.

In another embodiment, the method according to the present invention further comprises determining the level of one or more bacteria of the genus Blautia in the subject's gut.

In a further embodiment, the composition used in the method of the present invention is encapsulated.

### Dosage

The bacterial strains used in accordance with the present invention may be present from 10⁶ to 1014 CFU of bacteria/g of support, and more particularly from 10⁸ to 10¹² CFU of bacteria/g of support, preferably 109 to 10¹² CFU/g of support. By “support” is meant a composition, a food product, a food ingredient, a dietary supplement or a pharmaceutically acceptable composition.

Suitably, the bacterial strains of the genus Blautia used in accordance with the present invention may be administered at a dosage of from about 10⁶ to about 1014 CFU of microorganism/dose, preferably about 10⁸ to about 10¹² CFU of microorganism/dose and more preferably from about 109 to about 10¹¹ CFU of microorganism/dose. CFU stands for “colony-forming units”. By the term “per dose” it is meant that this amount of microorganism is provided to a subject either per day or per intake, preferably per day. For example, if the microorganisms are to be administered in a food product, for example in a yoghurt, then the yoghurt will preferably contain from about 10⁸ to 10¹² CFU of the microorganism. Alternatively, however, this amount of microorganism may be split into 5 multiple administrations each consisting of a smaller amount of microbial loading—so long as the overall amount of microorganism received by the subject in any specific time, for instance each 24-hour period, is from about 10⁶ to about 10¹² CFU of microorganism, preferably 10⁸ to about 10¹² CFU of microorganism and more preferably from about 10⁹ to about 10¹¹ CFU of microorganism. In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, the composition according to the present invention comprises at least 1×10⁶ colony forming units (CFUs) of the bacteria of the genus Blautia.

### Food Product

In one embodiment, the bacterial strains are used according to the invention in a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

When used as, or in the preparation of, a food, such as functional food, the bacteria of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the bacteria of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, vegetable milk, acidified soy/juice beverage, aseptic/retorted chocolate drink, bar mixes, beverage powders, calcium fortified soy/plain and chocolate milk, calcium fortified coffee beverage.

Advantageously, where the product is a food product, the bacterial strains should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment age.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Dietary Supplements

The compositions of the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help prevent or treat diseases.

### Medical Food

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Composition

The bacteria of the present invention may be used as—or in the preparation of—a pharmaceutical composition or formulation. Here, the term “pharmaceutical” is used in a broad sense—and covers pharmaceuticals for humans as well as pharmaceuticals for animals (i.e. veterinary applications).

In a preferred embodiment, the pharmaceutical acceptable composition is a medicament.

The pharmaceutical composition can be for therapeutic purposes—which may be curative or palliative or preventative in nature.

In a preferred embodiment of the present invention, the medicament is for oral administration.

A pharmaceutically acceptable composition or support may be for example a formulation or support in the form of creams, foams, gels, lotions, and ointments of compressed tablets, tablets, capsules, ointments, suppositories or drinkable solutions.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

The bacterial strains of the present invention may be used as pharmaceutical ingredients. Here, the composition may be the sole active component, or it may be at least one of a number (i.e. 2 or more) of active components.

The bacterial strains may be used according to the present invention in any suitable form—whether when alone or when present in a combination with other components or ingredients. Likewise, combinations comprising the bacteria of the present invention and other components and/or ingredients (i.e. ingredients—such as food ingredients, functional food ingredients or pharmaceutical ingredients) may be used in any suitable form.

The bacterial strains may be used according to the present invention in the form of solid or liquid preparations or alternatives thereof. Examples of solid preparations include, but are not limited to tablets, capsules, dusts, granules and powders which may be water dispersible, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the bacteria of the present invention are used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid 30 monoglycerides and diglycerides, petroethrai fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the bacteria of the present invention may be combined with various sweetening or flavouring agents, colouring matter or dyes, with emulsifying and/or suspending agents and with diluents such as water, propylene glycol and glycerin, and combinations thereof.

The forms may also include gelatine capsules; fibre capsules, fibre tablets etc.; or even fibre beverages.

In one aspect, the bacteria according to the present invention may be administered in an aerosol, for example by way of a nasal spray, for instance for administration to the respiratory tract.

### Prebiotics

In one embodiment, the bacterial strains and compositions of the present invention may further be combined or comprise one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal or skin microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat and unrefined barley.

Examples of suitable prebiotics include alginate, xanthan, pectin, locust bean gum (LBG), inulin, guar gum, galacto-oligosaccharide (GOS), fructo-oligosaccharide (FOS), polydextrose 10 (i.e. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, (human) milk oligosaccharides and all forms of resistant starches.

The combination of one or more of the bacterial strains according to the present invention and one or more fibres and/or prebiotics according to the present invention exhibits a synergistic effect in certain applications (i.e. an effect which is greater than the additive effect of the bacteria when used separately).

In one embodiment, the bacterial strains or a mixture thereof according to the present invention is used in combination with one or more fibres and/or prebiotic.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, 5 microcrystalline cellulose (MCC), milk oligosaccharide or betaine.

In a further aspect, the invention relates to a composition, food products, food ingredient, dietary supplements or a pharmaceutical acceptable composition comprising bacterial strains according to the present invention or a mixture thereof and one or more fibres and/or a prebiotic.

### Embodiments of the Invention

For the avoidance of doubt, some of the embodiments the present invention relates to are set out below: Embodiment 1. Bacterial strain of the Genus Blautia, wherein said strain has a 16S ribosomal RNA sequence with at least 98.8%, at least 99% or at least 99.5% sequence identity to 16S ribosomal RNA SEQ ID NO: 3.

Embodiment 2. The bacterial strain according to embodiment 1, wherein said strain has a 16S ribosomal RNA sequence identical to SEQ ID NO: 3.

Embodiment 3. Bacterial strain of the Genus Blautia, wherein said strain has a 16S ribosomal RNA sequence with at least 97.6%, at least 98% or at least 99% sequence identity to 16S ribosomal RNA SEQ ID NO: 4.

Embodiment 4. The bacterial strain according to embodiment 3, wherein said strain has a 16S ribosomal RNA sequence identical to SEQ ID NO: 4.

Embodiment 5. Bacterial strain of the Genus Blautia, wherein said strain has a 16S ribosomal RNA sequence with at least 99.95% sequence identity to 16S ribosomal RNA SEQ ID NO: 5.

Embodiment 6. The bacterial strain according to embodiment 5, wherein said strain has a has a 16S ribosomal RNA sequence identical to SEQ ID NO: 5.

Embodiment 7. Bacterial strain of the Genus Blautia, wherein said strain has a 16S ribosomal RNA sequence with at least 99.9% sequence identity to 16S ribosomal RNA SEQ ID NO: 6.

Embodiment 8. The bacterial strain according to embodiment 7, wherein said strain has a 16S ribosomal RNA sequence identical to SEQ ID NO: 6.

Embodiment 9. Bacterial strain of the genus Blautia, wherein the strain was deposited with the DSMZ on 9 Mar. 2021 under accession number DSM 33834 or has at least 80% nucleotide sequence identity to the nucleotide sequence of said deposited strain.

Embodiment 10. The bacterial strain according to embodiment 9, wherein the strain has a 16S ribosomal RNA sequence with at least 98.8%, at least 99% or at least 99.5% sequence identity to 16S ribosomal RNA SEQ ID NO: 3.

Embodiment 11. The bacterial strain according to embodiment 9, wherein said strain was deposited with the DSMZ on 9 Mar. 2021 under accession number DSM 33834.

Embodiment 12. Bacterial strain of the genus Blautia, wherein the strain was deposited with the DSMZ on 10 Feb. 2021 under accession number DSM 33790 or has at least 80% nucleotide sequence identity to the nucleotide sequence of said deposited strain.

Embodiment 13. The bacterial strain according to embodiment 12, wherein the strain has a 16S ribosomal RNA sequence with at least 97.6%, at least 98% or at least 99% sequence identity to 16S ribosomal RNA SEQ ID NO: 4.

Embodiment 14. Bacterial strain according to embodiment 12, wherein said strain was deposited with the DSMZ on 10 Feb. 2021 under accession number DSM 33790.

Embodiment 15. Bacterial strain of the genus Blautia, wherein the strain was deposited with the DSMZ on 10 Feb. 2021 under accession number DSM 33791 or has at least 80% nucleotide sequence identity to the nucleotide sequence of said deposited strain.

Embodiment 16. The bacterial strain according to embodiment 15, wherein the strain has a 16S ribosomal RNA sequence with at least 99.95% sequence identity to 16S ribosomal RNA SEQ ID NO: 5.

Embodiment 17. Bacterial strain according to embodiment 15, wherein said strain was deposited with the DSMZ on 10 Feb. 2021 under accession number DSM 33791.

Embodiment 18. Bacterial strain of the genus Blautia, wherein the strain was deposited with the DSMZ on 9 Mar. 2021 under accession number DSM 33833 or has at least 80% nucleotide sequence identity to the nucleotide sequence of said deposited strain.

Embodiment 19. The bacterial strain according to embodiment 18, wherein the strain has a 16S ribosomal RNA sequence with at least 99.9% sequence identity to 16S ribosomal RNA SEQ ID NO: 6.

Embodiment 20. The bacterial strain according to embodiment 18, wherein said strain was deposited with the DSMZ on 9 Mar. 2021 under accession number DSM 33833.

Embodiment 21. Use of a bacterial strain of the genus Blautia for inducing the production of interleukins IL-12 and/or IL-10.

Embodiment 22. The use according to embodiment 21, wherein the interleukins IL-12 and/or IL-10 induce an immunoboosting effect.

Embodiment 23. The use according to embodiment 21, wherein the interleukins IL-12 and/or IL-10 induce a T-helper 1 (Th1) type immunostimulation and/or an anti-inflammatory effect.

Embodiment 24. Bacterial strain of the genus Blautia for use as a medicament.

Embodiment 25. The bacterial strain according to embodiment 24, wherein said strain induces the production of interleukins IL-12 and/or IL-10.

Embodiment 26. The bacterial strain according to embodiment 25, wherein the interleukins IL-12 and/or IL-10 induce an immunoboosting effect.

Embodiment 27. The bacterial strain according to embodiment 25, wherein the interleukins IL-12 and/or IL-10 induce a T-helper 1 (Th1) type immunostimulation and/or an anti-inflammatory effect.

Embodiment 28. The bacterial strain of the genus Blautia according to any one of the embodiments 24-27 for use in preventing, treating and/or in reducing symptoms associated with a rhinovirus-induced common cold infection in a subject in need thereof.

Embodiment 29. The bacterial strain for use according to embodiment 28, wherein the symptoms associated with a rhinovirus-induced common cold infection are sneezing, nasal obstruction, nasal discharge, sore throat, cough, headache, chilliness and malaise.

Embodiment 30. The bacterial strain for use according to embodiment 24, wherein the bacteria was strain deposited with DSMZ on 10 Feb. 2021 under accession number DSM 33790.

Embodiment 31. The bacterial strain for use according to embodiment 24, wherein the bacteria of the genus Blautia are of the species Blautia obeum and/or Blautia faecis.

Embodiment 32. The bacterial strain for use according to embodiment 31, wherein the species Blautia obeum is strain deposited with DSMZ on 9 Mar. 2021 under accession number DSM 33834.

Embodiment 33. The bacterial strain for use according to embodiment 31, wherein the species Blautia faecis is strain deposited with DSMZ on 10 Feb. 2021 under accession number DSM 33791.

Embodiment 34. The bacterial strain for use according to embodiment 31, wherein the species Blautia faecis is strain deposited with DSMZ on 9 Mar. 2021 under accession number DSM 33833.

Embodiment 35. The bacterial strain for use according any one of the embodiments 28-34, wherein the bacteria are live bacteria.

Embodiment 36. A composition comprising bacteria of the genus Blautia for use as a medicament.

Embodiment 37. A composition comprising bacteria of the genus Blautia according to embodiment 36 for use in preventing, treating and/or reducing symptoms associated with a rhinovirus-induced common cold infection in a subject in need thereof.

Embodiment 38. The composition for use according to embodiment 37, wherein the symptoms associated with a rhinovirus-induced common cold infection are sneezing, nasal obstruction, nasal discharge, sore throat, cough, headache, chilliness and malaise.

Embodiment 39. The composition for use according to embodiment 36, wherein the bacteria is strain deposited with DSMZ on Feb. 10, 2021 under accession number DSM 33790.

Embodiment 40. The composition for user according to embodiment 36, wherein the bacteria are of the species Blautia obeum and/or Blautia faecis.

Embodiment 41. The composition for use according to embodiment 40, wherein the species Blautia obeum is strain deposited with DSMZ on 9 Mar. 2021 under accession number DSM 33834.

Embodiment 42. The composition for use according to embodiment 40, wherein the species Blautia faecis is strain deposited with DSMZ on 10 Feb. 2021 under accession number DSM 33791.

Embodiment 43. The composition for use according to embodiment 40, wherein the species Blautia faecis is strain deposited with DSMZ on 9 Mar. 2021 under accession number DSM 33833.

Embodiment 44. The composition for use according any one of the embodiments 36-43, wherein the bacteria are live bacteria.

Embodiment 45. The composition for use according to any one of the embodiments 36-44, wherein said composition is a food product, a food ingredient, a dietary supplement or a pharmaceutical acceptable composition.

Embodiment 46. A method of preventing, treating and/or reducing symptoms associated with a rhinovirus-induced common cold infection in a subject in need thereof, comprising treating said subject with a treatment regimen comprising administering a composition comprising an effective amount of bacteria of the genus Blautia.

Embodiment 47. The method according to embodiment 46, wherein the treatment regimen comprises oral administration of the composition.

Embodiment 48. The method according to embodiment 46, wherein the treatment regimen comprises rectal administration of the composition.

Embodiment 49. The method according to embodiment 46, wherein the bacteria of the genus Blautia is as defined in any one of embodiments 1-20.

Embodiment 50. The method according to any one of embodiments 46-49, wherein the treatment regimen comprises administering the composition for at least 1 week.

Embodiment 51. The method according to any one of embodiments 46-49, wherein the treatment regimen comprises administering the composition for at least 2 weeks.

Embodiment 52. The method according to any one of embodiments 46-51, wherein the treatment regimen comprises administering the composition at least two times per week.

Embodiment 53. The method according to any one of embodiments 46-52, wherein the method further comprises determining the level of one or more bacteria of the genus Blautia in the subject's gut.

Embodiment 54. The method according to any one of embodiments 46-52, wherein said composition comprises at least 1×10⁶ colony forming units (CFUs) of the bacteria of the genus Blautia.

Embodiment 55. The method according to any one of embodiments 46-54, wherein the composition is encapsulated.

### EXAMPLES

### Example 1: Discovery of Blautia Species for Reduced Cold Infection Symptoms

Clinical study. All work involving human subjects was approved by the Human Investigations Committee of the University of Virginia. The study was conducted in compliance with Good Clinical Practices and in accordance with the Declaration of Helsinki. A clinical study was conducted in healthy human adults which investigated the effect of experimental rhinovirus challenge on the faecal microbiota composition and potential associations between microbial taxa abundance and clinical outcomes, including daily symptom scores, illness duration, viral titer loads. On study day 0, faecal samples were collected at the clinic from the volunteers (n=229, 5 cohorts), after which the volunteers were challenged with approximately 100 tissue culture infectious dose 50 (TCID₅₀) of virus rhinovirus type 39 (RV-A39) by intranasal drops (Food and Drug Administration (FDA) IND #15241). The volunteers then visited the clinic daily for five days (study days 1-5) post-inoculation for the collection of specimens and symptom assessment. Symptom scoring was done according to the modified Jackson symptom score, where nasal obstruction, rhinorrhoea, sore throat, cough, sneezing, headache, myalgia, and chilliness were scored as 0-4 corresponding to absent, mild, moderate, severe or very severe.

The total symptom score corresponds to the added score for all symptoms. 16S rRNA amplicon sequencinq of faecal microbiota. Faecal samples were stored at −80° C. until processing. Microbial DNA was isolated from 200 mg of the faecal samples by mechanical lysis (Precellys 24 Homogenizer, Bertin Instruments) followed by extraction using the Applied Biosystem AM1840 MagMAX™ Total Nucleic Acid Isolation kit on the MagMAX™ Express 96 DNA extraction robot (Thermo Fisher Scientific). Microbiota taxonomic profiles were generated using 16S rRNA amplicon sequencing (Caporaso et al., 2012). The V4 variable region of the 16S rRNA gene was amplified by barcoded PCR primers 515F (5′-GTGCCAGCMGCCGCGGTAA) and 806R (5′-GGACTACHVGGGTWTCTAAT) with the following conditions: denaturation at 95° C. for 3 min followed by 30 cycles of denaturation at 95° C. for 45s, annealing at 55° C. for 60s and extension at 72° C. for 90s, and a final extension at 72° C. for 10 min. PCR products were purified, normalized by DNA concentration and sequenced with 2 replicate MiSeq V2 lanes with 2×250nt (Illumina).

Sequencing data were analysed using the QIIME2 pipeline (Bolyen et al., 2019). Reads were demultiplexed and paired using ‘qiime demux,’ and ‘qiime dada2’ was used to error-model and correct the Illumina reads, including denoising, dereplication and removal of chimeras using the ‘consensus’ method (Callahan et al., 2016). Taxonomy was assigned to the amplicon sequence variants (ASVs) using ‘q2-feature-classifier’ (classify-sklearn) trained on the V4 region of sequences contained in the Greengenes database (v. 13.8) (DeSantis et al., 2006).

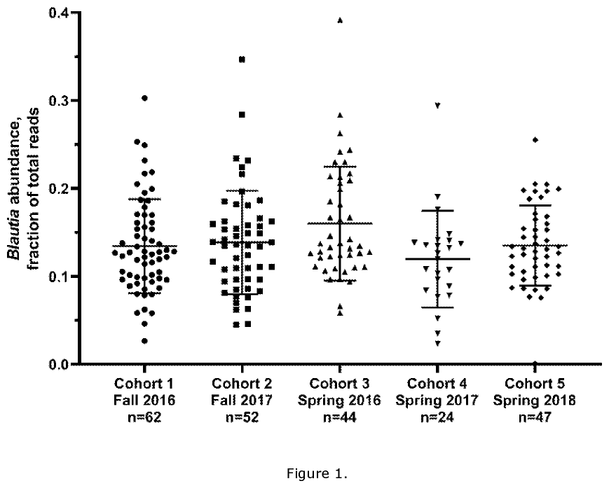

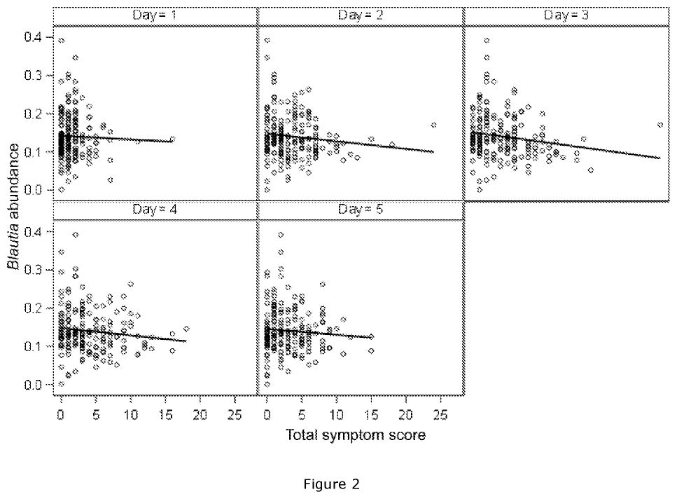

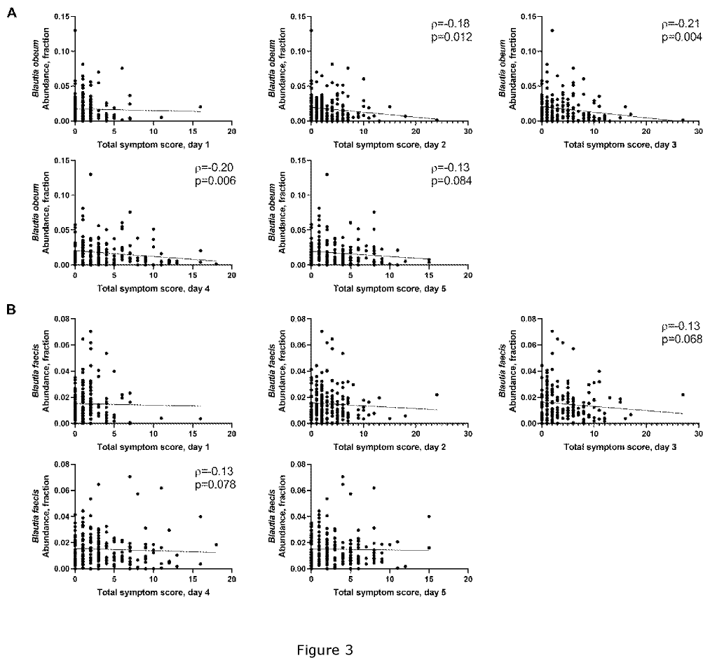

Blautia was the most abundant genera detected in the microbiota from the faecal samples (FIG. 1); therefore, potential associations with clinical outcomes including duration of illness and clinical symptom score were investigated. Total symptom score was associated with Blautia abundance (overall taxa effect p=0.022, Poisson Regression), where subjects with higher Blautia abundance at study day 0 had lower symptom scores on study days 1-5 (FIG. 2). An analysis of all ASVs with a taxonomic assignment to the genus Blautia further identified two ASVs corresponding to the species Blautia obeum and Blautia faecis with correlations to the clinical symptom scores (Table 1). A higher abundance of Blautia obeum (SEQ ID NO:1) at study day 0 corresponded to lower daily symptom scores at study days 2-5, and a higher abundance of Blautia faecis (SEQ ID NO:2) corresponded to lower daily symptom scores at study days 3 and 4 (FIG. 3).

TABLE 1
16S rRNA gene sequences from V4 variable
region for Blautia obeum and Blautia faecis
ASVs detected from faecal samples in
human clinical study.

| SEQ | | |
|---|---|---|
| ID | | |
| NO: | Bacteria | Nucleotide sequence (253 nt) |
| 1 | Blautia | TACGTAGGGGGCAAGCGTTATCCGGATTTA |
| | obeum | CTGGGTGTAAAGGGAGCGTAGACGGACTGG |
| | | CAAGTCTGATGTGAAAGGCGGGGGCTCAAC |
| | | CCCTGGACTGCATTGGAAACTGTTAGTCTT |
| | | GAGTGCCGGAGAGGTAAGCGGAATTCCTAG |
| | | TGTAGCGGTGAAATGCGTAGATATTAGGAG |
| | | GAACACCAGTGGCGAAGGCGGCTTACTGGA |
| | | CGGTAACTGACGTTGAGGCTCGAAAGCGTG |
| | | GGGAGCAAACAGG |
| 2 | Blautia | TACGTAGGGGGCAAGCGTTATCCGGATTTA |
| | faecis | CTGGGTGTAAAGGGAGCGTAGACGGCGCAG |
| | | CAAGTCTGATGTGAAAGGCAGGGGCTTAAC |
| | | CCCTGGACTGCATTGGAAACTGCTGTGCTT |
| | | GAGTGCCGGAGGGGTAAGCGGAATTCCTAG |
| | | TGTAGCGGTGAAATGCGTAGATATTAGGAG |
| | | GAACACCAGTGGCGAAGGCGGCTTACTGGA |
| | | CGGTAACTGACGTTGAGGCTCGAAAGCGTG |
| | | GGGAGCAAACAGG |

### Example 2: Isolation and Culturing of Blautia Strains for Probiotic Use

Blautia isolates were recovered from faecal samples of healthy human adult donors. Diluted faecal material was plated onto BHIB agar plates and incubated for 24 hours under anaerobic conditions. BHIB: Brain Heart Infusion agar supplemented with 10% Sheep Blood (purchased commercially, BD 221843). BHIS was used for growth in liquid broth medium. BHIS: Brain Heart Infusion supplemented with yeast extract, Vitamin K1 and hemin. All microbiological culturing was performed in an anaerobic chamber using a mixed gas of N2/CO2/H2 (85/10/5 Taxonomic identification of the isolates was confirmed using PS rRNA sequencing. An aliquot of cell culture growth in BHIS was diluted 1:100 with sterile water and used as the template in a 16S PGR reaction. PGR primers used to amplify the 16S gene were: 8F: AGA GTT TGA TYM TGG GTG and 1492R: GGG TTA GGT TGT TAG GAG TT under PGR reaction conditions that were standard for polymerase Q5. Aliquots of the PGR reactions were run on an agarose gel to confirm the presence of a PGR product of expected size, and then purified using the ExoSAP-IT Express PGR Cleanup Kit. The samples were sent for Sanger sequencing using a third-party vendor.

Four Blautia strains were recovered the human faecal material (Table 2) that corresponded to SEQ ID NO:1 and SEQ ID NO:2 (Table 1).

TABLE 2
16S rRNA gene sequences for Blautia obeum and
Blautia faecis probiotic strains.

| | | | | | Pairwise |
|---|---|---|---|---|---|
| SEQ | | | | | similarity |
| ID | | DSMZ | | Type | to Type |
| NO: | Strain ID | Deposit | Nucleotide sequence | Strain | strain (%) |
| 3 | Blautia | DSM33834 | AGCTGGTTTCTAGTGGCGGACGGGT | Blautia | 98.7 |
| | obeum | | GAGTAACGCGTGGGTAACCTGCCTT | obeum | |
| | P75_3586 | | ATACAGGGGGATAACAACCAGAAAT | VPI B3- | |
| | DGCC13628 | | GGTTGCTAATACCGCATAAGCGCAC | 21 | |
| | | | AGGACCGCATGGTCCGGTGTGAAAA | | |
| | | | ACTCCGGTGGTATAAGATGGACCCG | | |
| | | | CGTTGGATTAGCTAGTTGGCAGGGT | | |
| | | | AACGGCCTACCAAGGCGACGATCCA | | |
| | | | TAGCCGGCCTGAGAGGGTGAACGGC | | |
| | | | CACATTGGGACTGAGACACGGCCCA | | |
| | | | GACTCCTACGGGAGGCAGCAGTGGG | | |
| | | | GAATATTGCACAATGGGGGAAACCC | | |
| | | | TGATGCAGCGACGCCGCGTGAAGGA | | |
| | | | AGAAGTATCTCGGTATGTAAACTTC | | |
| | | | TATCAGCAGGGAAGATAGTGACGGT | | |
| | | | ACCTGACTAAGAAGCCCCGGCTAAC | | |
| | | | TACGTGCCAGCAGCCGCGGTAATAC | | |
| | | | GTAGGGGGCAAGCGTTATCCGGATT | | |
| | | | TACTGGGTGTAAAGGGAGCGTAGAC | | |
| | | | GGACTGGCAAGTCTGATGTGAAAGG | | |
| | | | CGGGGGCTCAACCCCTGGACTGCAT | | |
| | | | TGGAAACTGTTAGTCTTGAGTGCCG | | |
| | | | GAGAGGTAAGCGGAATTCCTAGTGT | | |
| | | | AGCGGTGAAATGCGTAGATATTAGG | | |
| | | | AGGAACACCAGTGGCGAAGGCGGCT | | |
| | | | TACTGGACGGTAACTGACGTTGAGG | | |
| | | | CTCGAAAGCGTGGGGAGCAAACAGG | | |
| | | | ATTAGATACCCTGGTAGTCCACGCC | | |
| | | | GTAAACGATGAATACTAGGTGTTGG | | |
| | | | GGAGCAAAGCTCTTCGGTGCCGCCG | | |
| | | | CAAACGCATTAAGTATTCCACCTGG | | |
| | | | GGAGTACGTTCGCAAGAATGAAACT | | |
| | | | CAAAGGAATTGACGGGGACCCGCAC | | |
| | | | AAGCGGTGGAGCATGTGGTTTAATT | | |
| | | | CGAAGCAACGCGAAGAACCTTACCA | | |
| | | | AGTCTTGACATCCCTCTGACCGACT | | |
| | | | CTTAACCGAGTCTTTCCTTCGGGAC | | |
| | | | AGAGGAGACAGGTGGTGCATGGTTG | | |
| | | | TCGTCAGCTCGTGTCGTGAGATGTT | | |
| | | | GGGTTAAGTCCCGCAACGAGCGCAA | | |
| | | | CCCCTATCCCCAGTAGCCAGCA | | |
| | Blautia sp. | DSM33790 | AACCTATTATTGAAACTTCGGTCGA | Blautia | 97.5 |
| 4 | P22_1012 | | TTTAATTTGTTTCTAGTGGCGGACG | obeum | |
| | DGCC13627 | | GGTGAGTAACGCGTGGGTAACCTGC | VPI B3- | |
| | | | CCTATACAGGGGGATAACAACCAGA | 21 | |
| | | | AATGGTTGCTAATACCGCATAAGCG | | |
| | | | CACAGGACCGCATGGTCCAGTGTGA | | |
| | | | AAAACTCCGGTGGTATAGGATGGAC | | |
| | | | CCGCGTTGGATTAGCTTGTTGGTGG | | |
| | | | GGTAACGGCCCACCAAGGCGACGAT | | |
| | | | CCATAGCCGGCCTGAGAGGGTGAAC | | |
| | | | GGCCACATTGGGACTGAGACACGGC | | |
| | | | CCAGACTCCTACGGGAGGCAGCAGT | | |
| | | | GGGGAATATTGCACAATGGGGGAAA | | |
| | | | CCCTGATGCAGCGACGCCGCGTGAA | | |
| | | | GGAAGAAGTATCTCGGTATGTAAAC | | |
| | | | TTCTATCAGCAGGGAAGATAGTGAC | | |
| | | | GGTACCTGACTAAGAAGCCCCGGCT | | |
| | | | AACTACGTGCCAGCAGCCGCGGTAA | | |
| | | | TACGTAGGGGGCAAGCGTTATCCGG | | |
| | | | ATTTACTGGGTGTAAAGGGAGCGTA | | |
| | | | GACGGACTGGCAAGTCTGATGTGAA | | |
| | | | AGGCGGGGGCTCAACCCCTGGACTG | | |
| | | | CATTGGAAACTGTTAGTCTTGAGTG | | |
| | | | CCGGAGAGGTAAGCGGAATTCCTAG | | |
| | | | TGTAGCGGTGAAATGCGTAGATATT | | |
| | | | AGGAGGAACACCAGTGGCGAAGGCG | | |
| | | | GCTTACTGGACGGTAACTGACGTTG | | |
| | | | AGGCTCGAAAGCGTGGGGAGCAAAC | | |
| | | | AGGATTAGATACCCTGGTAGTCCAC | | |
| | | | GCCGTAAACGATGAATACTAGGTGT | | |
| | | | TGGGGAGCAAAGCTCTTCGGTGCCG | | |
| | | | CCGCAAACGCATTAAGTATTCCACC | | |
| | | | TGGGGAGTACGTTCGCAAGAATGAA | | |
| | | | ACTCAAAGGAATTGACGGGGACCCG | | |
| | | | CACAAGCGGTGGAGCATGTGGTTTA | | |
| | | | ATTCGAAGCAACGCGAAGAACCTTA | | |
| | | | CCAAGTCTTGACATCCCTCTGACCG | | |
| | | | ATCCGTAACGGGATCTTTCCTTCGG | | |
| | | | GACAGGGGAGACAGGTGGTGCATGG | | |
| | | | TTGTCGTCAGCTCGTGTCGTGAGAT | | |
| | | | GTTGGGTTAAGTCCCGCAACGAGCG | | |
| | | | CAACCCCTATCCCCAGTAGCCAGCG | | |
| | | | GTTTGGCCGGGCACTCTGAGGAGAC | | |
| | | | TGCCAGGGATAACCTGGAGGAAGGC | | |
| | | | GGGGATGACGTCAAATCATCATGCC | | |
| | | | CCTTATGATTTGGGCTACACACGTG | | |
| | | | CTACAATGGCGTAAACAAAGGGAAG | | |
| | | | CGAGCCTGCGAAGGTAA | | |
| 5 | Blautia | DSM33791 | CTGGGGGATAACAGCCAGAAATGAC | Blautia | 99.9 |
| | faecis | | TGCTAATACCGCATAAGCGCACAGA | faecis | |
| | P73_3472 | | ACCGCATGGTTCCGTGTGAAAAACT | M25 | |
| | DGCC13629 | | CCGGTGGTATAAGATGGACCCGCGT | | |
| | | | TGGATTAGCTAGTTGGCAGGGCAGC | | |
| | | | GGCCTACCAAGGCGACGATCCATAG | | |
| | | | CCGGCCTGAGAGGGTGAACGGCCAC | | |
| | | | ATTGGGACTGAGACACGGCCCAGAC | | |
| | | | TCCTACGGGAGGCAGCAGTGGGGAA | | |
| | | | TATTGCACAATGGGGGAAACCCTGA | | |
| | | | TGCAGCGACGCCGCGTGAAGGAAGA | | |
| | | | AGTATCTCGGTATGTAAACTTCTAT | | |
| | | | CAGCAGGGAAGATAATGACGGTACC | | |
| | | | TGACTAAGAAGCCCCGGCTAACTAC | | |
| | | | GTGCCAGCAGCCGCGGTAATACGTA | | |
| | | | GGGGGCAAGCGTTATCCGGATTTAC | | |
| | | | TGGGTGTAAAGGGAGCGTAGACGGC | | |
| | | | GCAGCAAGTCTGATGTGAAAGGCAG | | |
| | | | GGGCTTAACCCCTGGACTGCATTGG | | |
| | | | AAACTGCTGTGCTTGAGTGCCGGAG | | |
| | | | GGGTAAGCGGAATTCCTAGTGTAGC | | |
| | | | GGTGAAATGCGTAGATATTAGGAGG | | |
| | | | AACACCAGTGGCGAAGGCGGCTTAC | | |
| | | | TGGACGGTAACTGACGTTGAGGCTC | | |
| | | | GAAAGCGTGGGGAGCAAACAGGATT | | |
| | | | AGATACCCTGGTAGTCCACGCCGTA | | |
| | | | AACGATGAATACTAGGTGTCAGGGA | | |
| | | | GCACAGCTCTTTGGTGCCGCCGCAA | | |
| | | | ACGCATTAAGTATTCCACCTGGGGA | | |
| | | | GTACGTTCGCAAGAATGAAACTCAA | | |
| | | | AGGAATTGACGGGGACCCGCACAAG | | |
| | | | CGGTGGAGCATGTGGTTTAATTCGA | | |
| | | | AGCAACGCGAAGAACCTTACCAAAT | | |
| | | | CTTGACATCCCTCTGACCGGGACTT | | |
| | | | AACCGTCCCTTTCCTTCGGGACAGG | | |
| | | | GGAGACAGGTGGTGCATGGTTGTCG | | |
| | | | TCAGCTCGTGTCGTGAGATGTTGGG | | |
| | | | TTAAGTCCCGCAACGAGCGCAACCC | | |
| | | | CTATCCTTAGTAGCCAGCACGCAGT | | |
| | | | GGTGGGCACTCTGAGGAGACTGCCA | | |
| | | | GGGATAACCTGGAGGAAGGCGGGGA | | |
| | | | TGACGTCAAATCATCATGCCCCTTA | | |
| | | | TGATTTGGGCTACACACGTGCTACA | | |
| | | | ATGGCGTAAACAAAGGGAAGCGAAC | | |
| | | | CCGCGAGGGTGGGCAAATCTCAAAA | | |
| | | | ATAACGTCCCAGTTC | | |
| 6 | Blautia | DSM33833 | ACTTTATTGAAACTTCGGTGGATTTA | Blautia | 99.8 |
| | faecis | | ATTTGTTTCTAGTGGCGGACGGGTG | faecis | |
| | P129_7565 | | AGTAACGCGTGGGTAACCTGCCTTA | M25 | |
| | DGCC13626 | | TACTGGGGGATAACAGCCAGAAATG | | |
| | | | ACTGCTAATACCGCATAAGCGCACA | | |
| | | | GAACCGCATGGTTCCGTGTGAAAAA | | |
| | | | CTCCGGTGGTATAAGATGGACCCGC | | |
| | | | GTTGGATTAGCTAGTTGGCAGGGCA | | |
| | | | GCGGCCTACCAAGGCGACGATCCAT | | |
| | | | AGCCGGCCTGAGAGGGTGAACGGCC | | |
| | | | ACATTGGGACTGAGACACGGCCCAG | | |
| | | | ACTCCTACGGGAGGCAGCAGTGGGG | | |
| | | | AATATTGCACAATGGGGGAAACCCT | | |
| | | | GATGCAGCGACGCCGCGTGAAGGAA | | |
| | | | GAAGTATCTCGGTATGTAAACTTCT | | |
| | | | ATCAGCAGGGAAGATAATGACGGTA | | |
| | | | CCTGACTAAGAAGCCCCGGCTAACT | | |
| | | | ACGTGCCAGCAGCCGCGGTAATACG | | |
| | | | TAGGGGGCAAGCGTTATCCGGATTT | | |
| | | | ACTGGGTGTAAAGGGAGCGTAGACG | | |
| | | | GCGCAGCAAGTCTGATGTGAAAGGC | | |
| | | | AGGGGCTTAACCCCTGGACTGCATT | | |
| | | | GGAAACTGCTGTGCTTGAGTGCCGG | | |
| | | | AGGGGTAAGCGGAATTCCTAGTGTA | | |
| | | | GCGGTGAAATGCGTAGATATTAGGA | | |
| | | | GGAACACCAGTGGCGAAGGCGGCTT | | |
| | | | ACTGGACGGTAACTGACGTTGAGGC | | |
| | | | TCGAAAGCGTGGGGAGCAAACAGGA | | |
| | | | TTAGATACCCTGGTAGTCCACGCCG | | |
| | | | TAAACGATGAATACTAGGTGTCAGG | | |
| | | | GAGCACAGCTCTTTGGTGCCGCCGC | | |
| | | | AAACGCATTAAGTATTCCACCTGGG | | |
| | | | GAGTACGTTCGCAAGAATGAAACTC | | |
| | | | AAAGGAATTGACGGGGACCCGCACA | | |
| | | | AGCGGTGGAGCATGTGGTTTAATTC | | |
| | | | GAAGCAACGCGAAGAACCTTACCAA | | |
| | | | ATCTTGACATCCCTCTGACCGGGAC | | |
| | | | TTAACCGTCCCTTTCCTTCGGGACA | | |
| | | | GGGGAGACAGGTGGTGCATGGTTGT | | |
| | | | CGTCAGCTCGTGTCGTGAGATGTTG | | |
| | | | GGTTAAGTCCCGCAACGAGCGCAAC | | |
| | | | CCCTATCCTTAGTAGCCAGCACGTA | | |
| | | | ATGGTGGGCACTCTGAGGAGACTGC | | |
| | | | CAGGGATAACCTGGAGGAAGGCGGG | | |
| | | | GATGACGTCAAATCATCATGCCCCT | | |
| | | | TATGATTTGGGCTACACACGTGCTA | | |
| | | | CAATGGCGTAAACAAAGGGAAGCGA | | |
| | | | ACCCGCGA | | |

### Example 3: Immune Cell Stimulation Assays

Blautia sp. strains were grown to logarithmic growth phase, collected by centrifugation, washed once with phosphate-buffered saline (PBS) and suspended to cell culture medium. The optical density (OD)600 was adjusted to correspond to bacteria:host cell ratio of 10:1. Monocytes were purified from freshly collected leukocyte-rich buffy coats obtained from healthy blood donors through the Finnish Red Cross Blood Service. The use of human blood was approved by the Ethics Committee of the Hospital District of Helsinki and Uusimaa, Finland. Human peripheral blood mononuclear cells (PBMC) were isolated by density gradient centrifugation followed by purification of monocytes with CD14+ magnetic beads. To obtain macrophages purified monocytes were plated on 24 well plates 3×10⁵ cells/well and cultured for 7 days in Macrophage-SFM (Gibco, Life Technologies, Grand Island, NY, USA) with recombinant human GM-CSF (Miltenyi Biotech) 1000 IU/ml and 1% Antibiotic-Antimycotic. To differentiate monocytes into immature dendritic cells, monocytes were plated on 12 well plates 5×10⁵ cells/well (Falcon, Corning, NY, USA) and cultured for 7 d in RPMI-1640 (Sigma) supplemented with 1% Antibiotic-Antimycotic, 10% fetal bovine serum (FBS), IL-4 (400 IU/ml) and GM-CSF (1000 IU/ml).

Cells from four blood donors were stimulated with Blautia species for 24 h (macrophages) or 48 h (dendritic cells). PBMCs without stimulation were used as a control.

Cell culture supernatants from macrophages and dendriti cell cultures were analyzed for IL-10 and IL-12 by Quanterix multiplex enzyme-linked immunosorbent assay (Quanterix, Inc., Billerica, MA, USA). Results were analyzed with CiraSoft software (Quanterix).

### Statistical Analysis

The data were log 2-transformed prior to analysis. The data were analyzed using a linear model that included the main effects of the treatment and the donor (2-way ANOVA). Pairwise-comparisons between the treatments were performed using contrasts of estimated marginal means. The fit of the models was checked by inspecting the normality of the model residuals. All p-values from all fitted models were collected together and corrected for family-wise error rate using the Holm-Bonferroni method. All analyses were performed using software R version 4.1.3.

### Results

Table 1 shows the macrophage response to Blautia strains (Bf=Blautia faecis; Bs=Blautia sp.; Bo=Blautia obeum; CI=Confidence Interval).

TABLE 1

| | | Ratio of | | | |
|---|---|---|---|---|---|
| Cytokine | Comparison | cytokines | 95% CI | p-value | p-adj* |
| IL-10 | Bf P129_7567/Ctrl | 39.23 | (17.22-89.38) | <0.001 | <0.001 |
| | Bf P73_3472/Ctrl | 12.01 | (5.27-27.37) | <0.001 | <0.001 |
| | Bs P22_1012/Ctrl | 8.55 | (3.75-19.47) | <0.001 | <0.001 |
| | Bo P75_3586/Ctrl | 13.94 | (6.12-31.75) | <0.001 | <0.001 |
| IL-12 | Bf P129_7567/Ctrl | 10.40 | (4.74-22.83) | <0.001 | <0.001 |
| | Bf P73_3472/Ctrl | 6.15 | (2.8-13.49) | <0.001 | <0.001 |
| | Bs P22_1012/Ctrl | 2.20 | (1-4.83) | 0.049 | 0.049 |
| | Bo P75_3586/Ctrl | 3.40 | (1.55-7.47) | 0.004 | 0.009 |

*adjusted p-value using Holm-Bonferroni method

Table 2 shows the dendritic cell response to Blautia strains. Bf=Blautia faecis; Bs=Blautia sp.; Bo=Blautia obeum; CI=Confidence Interval.

TABLE 2

| | | Ratio of | | | |
|---|---|---|---|---|---|
| Cytokine | Comparison | cytokines | 95% CI | p-value | p-adj* |
| IL-10 | Bf P129_ | 4.18 | (2.16-8.08) | <0.001 | <0.001 |
| | 7567/Ctrl | | | | |
| | Bf P73_ | 4.67 | (2.41-9.03) | <0.001 | <0.001 |
| | 3472/Ctrl | | | | |
| | Bs P22_ | 6.29 | (3.25-12.17) | <0.001 | <0.001 |
| | 1012/Ctrl | | | | |
| | Bo P75_ | 3.23 | (1.67-6.25) | 0.002 | 0.003 |
| | 3586/Ctrl | | | | |
| | Bf P129_ | 99.80 | (42.71-233.22) | <0.001 | <0.001 |
| | 7567/Ctrl | | | | |
| IL-12 | Bf P73_ | 13.97 | (5.98-32.65) | <0.001 | <0.001 |
| | 3472/Ctrl | | | | |
| | Bs P22_ | 24.51 | (10.49-57.27) | <0.001 | <0.001 |
| | 1012/Ctrl | | | | |
| | Bo P75_ | 132.34 | (56.63-309.24) | <0.001 | <0.001 |
| | 3586/Ctrl | | | | |

*adjusted p-value using Holm-Bonferroni method

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. The method according to claim 28, wherein said strain has a 16S ribosomal RNA sequence with at least 98.8% sequence identity to 16S ribosomal RNA SEQ ID NO: 3.

2. The method according to claim 1, wherein said strain has a 16S ribosomal RNA sequence identical to SEQ ID NO: 3.

3. The method according to claim 28, wherein said strain has a 16S ribosomal RNA sequence with at least 97.6% sequence identity to 16S ribosomal RNA SEQ ID NO: 4.

4. The method according to claim 3, wherein said strain has a 16S ribosomal RNA sequence identical to SEQ ID NO: 4.

5. The method according to claim 28, wherein said strain has a 16S ribosomal RNA sequence with at least 99.95% sequence identity to 16S ribosomal RNA SEQ ID NO: 5.

6. The method according to claim 5, wherein said strain has a has a 16S ribosomal RNA sequence identical to SEQ ID NO: 5.

7. The method according to claim 28, wherein said strain has a 16S ribosomal RNA sequence with at least 99.9% sequence identity to 16S ribosomal RNA SEQ ID NO: 6.

8. The method according to claim 7, wherein said strain has a 16S ribosomal RNA sequence identical to SEQ ID NO: 6.

9. The method according to claim 28, wherein the strain was deposited with the DSMZ on 9 Mar. 2021 under accession number DSM 33834 or has at least 80% nucleotide sequence identity to the nucleotide sequence of said deposited strain.

10. (canceled)

11. The method according to claim 9, wherein said strain was deposited with the DSMZ on 9 Mar. 2021 under accession number DSM 33834.

12. The method according to claim 28, wherein the strain was deposited with the DSMZ on 10 Feb. 2021 under accession number DSM 33790 or has at least 80% nucleotide sequence identity to the nucleotide sequence of said deposited strain.

13. (canceled)

14. The method according to claim 12, wherein said strain was deposited with the DSMZ on 10 Feb. 2021 under accession number DSM 33790.

15. The method according to claim 28, wherein the strain was deposited with the DSMZ on 10 Feb. 2021 under accession number DSM 33791 or has at least 80% nucleotide sequence identity to the nucleotide sequence of said deposited strain.

16. (canceled)

17. The method according to claim 15, wherein said strain was deposited with the DSMZ on 10 Feb. 2021 under accession number DSM 33791.

18. The method according to claim 28, wherein the strain was deposited with the DSMZ on 9 Mar. 2021 under accession number DSM 33833 or has at least 80% nucleotide sequence identity to the nucleotide sequence of said deposited strain.

19. (canceled)

20. The method according to claim 18, wherein said strain was deposited with the DSMZ on 9 Mar. 2021 under accession number DSM 33833.

21. A method for inducing the production of interleukins IL-12 and/or IL-10 in need thereof, wherein the method comprising administering an effective amount of a bacterial strain of the genus Blautia to the subject.

22-24. (canceled)

25. The method according to claim 28, wherein said strain induces the production of interleukins IL-12 and/or IL-10 in the subject.

26. The method according to claim 25, wherein the interleukins IL-12 and/or IL-10 induce an immunoboosting effect in the subject.

27. The method according to claim 25, wherein the interleukins IL-12 and/or IL-10 induce a T-helper 1 (Th1) type immunostimulation and/or an anti-inflammatory effect in the subject.

28. A method of in preventing, treating and/or in reducing symptoms associated with a rhinovirus-induced common cold infection in a subject in need thereof, wherein the method comprises administering an effective amount of a bacterial strain of the genus Blautia to the subject.

29. The method according to claim 28, wherein the symptoms associated with a rhinovirus-induced common cold infection are selected from sneezing, nasal obstruction, nasal discharge, sore throat, cough, headache, chilliness and malaise.

30. (canceled)

31. The method according to claim 28, wherein the bacteria of the genus Blautia are of the species Blautia obeum and/or Blautia faecis.

32-34. (canceled)
